(19) United States
(12) Patent Application Publication
(10) Pub. No.: US 2022/0257868 A1
(43) Pub. Date: Aug. 18, 2022

(54) Medication delivery device

(71) Applicant: Eric Yan, San Francisco, CA (US)

(72) Inventor: Eric Yan, San Francisco, CA (US)

(21) Appl. No.: 17/175,057

(22) Filed: Feb. 12, 2021

(51) Int. Cl. A61M 5/315; A61M 5/24; G16H 20/17; G16H 10/60

(52) U.S. Cl. CPC A61M 5/31546; A61M 5/24; A61M 2005/3128; G16H 10/60; G16H 20/17

(57) ABSTRACT

The disclosure is directed to a medication delivery device and a removable elongate member and cartridge system, and methods of use thereof.

The medication delivery device is designed to be programmed by physicians or other health care personnel who enter medication orders into the computerized physician order entry (CPOE) of the electronic health record (EHR) in the usual fashion. These commands are then used to program the medication delivery device. The medication delivery device uses standard vials as cartridges to administer medication to the patient, syringe, or similar device.

### CROSS REFERENCE TO RELATED APPLICATIONS

### TECHNICAL FIELD

This disclosure relates to a medication delivery device and an elongate member and fluid cartridge system and methods of use thereof, such as the delivery of fluids in medical settings.

### BACKGROUND

The conventional method of preparing parenteral, or non-oral, medications de novo for patients is tedious, time-consuming, and error-prone. This method requires, among other things, drawing medication from a vial into a syringe. Once in a syringe, medication is administered by hand or pump. When given by hand, these medications require 35 seconds and 41 steps to prepare and administer (1). Human error can be attributed to over 80% of medication errors by using conventional method (2). Additional time and steps are required for medication to be given by infusion or syringe pumps, and these can introduce errors not found from giving medication by hand (3).

### Medication Delivery Device

The medication delivery device is designed to assist in the preparation and administration of parenteral medications. The medication delivery device administers parental medication packaged in vials into the body including intravenous, epidural, intrathecal, among others, syringe, or similar device.

### Automatic Loading of Vials

The medication delivery device uses vials as pre-filled fluid cartridges that are placed into fluid cartridge holders, which have an L shaped hingeable door that is normally open when they are empty and closes when cartridge is placed into them. The fluid cartridge holders and L shaped hingeable door are made of metal or magnets which are held firmly in place by turning on an electromagnet underneath fluid cartridge holder. The electromagnet or motor turns the fluid cartridge holders or pre-filled fluid cartridges to present the fluid cartridges to the camera. The electromagnet with or without a solenoid attached to fluid cartridge holders reverses polarity to push fluid cartridge holders or pre-filled fluid cartridges onto the spikes of the tubing.

### Medication Delivery Device Programming

The medication delivery device is programmed by physicians and other health care personnel by entering a medication order into the CPOE of the EHR. Modification of the prescription may be suggested by clinical decision support system (CDSS).

Because the CPOE is a centralized repository of patient data, the medication delivery device reliably and faithfully receives data that can be used to administer medication. At present, nurses interpret the medication order and administer medication by hand or manually reenter the order into the syringe or infusion pump.

The medication delivery device retrieves three groups of information from the CPOE: demographic, laboratory, and prescription information.

Demographic information includes, among other things, patient name, birthdate, and measured body weight. Laboratory information includes pregnancy, creatinine, and international normalized ratio (INR). Prescription information is taken from the CPOE of the EHR and includes medication name, dosage, and frequency. Any number of safeguards can then be implemented by the CDSS to modify the prescription based on laboratory information to adjust the medication dose due to allergies, drug-drug interactions (DDI), kidney and liver function, among others. Additional doses of medication can be automatically scheduled. This information is processed by the medication delivery device to calculate the precise dose to give to patients or dispense into a syringe or similar device to give to a patient.

For example, a physician uses the EHR to enter the dose of morphine into the CPOE. The dose is reduced by the CDSS for renal failure. The volume of medication is 2.5 milliliters and time is 10 seconds and is sent to the medication delivery device to be administered to the patient or dispensed into a syringe or similar device to give to a patient.

The medication delivery device retrieves the medication order from the CPOE of the EHR by software or other means, which is typically written as an order sentence. For morphine, for example, it is written as: morphine 2.5-5 mg q3-4hr PRN infused intravenously over 4-5 minutes.

Software of the medication delivery device separates the order sentence into its four main component parts of name, dose, frequency, and time in the table below:

| Name | Dose | Frequency | Time |
|---|---|---|---|
| Morphine | 2.5-5 mg | q3-4 hr | 4-5 minutes |

Additional separation by software converts the name into the National Drug Code (NDC) and frequency into a number and separates the components of the dose and time into its number and units.

| | | Number- | Number- | | | Frequency | Time- | Time- |
|---|---|---|---|---|---|---|---|---|
| Name | NDC | low | high | Units | Frequency | number | low | high |
| Morphine | 0409-3814 | 2.5 | 5 | mg | q3-4 hr | 9174 | 4 | 5 |

In this fashion, the medication delivery device can understand the volume of medication to dispense over time.

Data variables related to the delivery of medication may be included including maximum number of doses. Other data may be dependent on the patient such as blood pressure which may be used to titrate vasopressors, bispectral index (BIS) for sedatives, creatinine for opioids, and INR for anticoagulants.

### Bar Code Medication Administration (BCMA)

Once it is programmed by the CPOE of the EHR, the medication delivery device awaits the preparation of medication. The process of preparing medication with the medication delivery device involves health care personnel placing a vial onto the spike of an administration set of the medication delivery device. A camera is positioned in front of the barcode of an incoming vial. The process of preparing medication has been described in 11 steps (steps 12-22) (1), and 28 steps (steps 1-11, 23-30, 32, 34-41) required by convention method are rendered unnecessary (1).

Once the vial is placed into the medication delivery device, a camera of the medication delivery device, an implementation of BCMA, reads the NDC from the barcode, which is machine-readable data. The NDC, a unique 10-digit, 3-segment number, provides information on final marketed drugs submitted to FDA and serves as a universal product identifier for medication. Once scanned, the medication delivery device identifies contents in the cartridge by accessing a remote server over a network by querying a database from the FDA (https://www.fda.gov/Drugs/InformationOnDrugs/ucm142438.htm) that provides information about the product including the medication name, concentration, and volume to verify that the proper medication is entered into the medication delivery device. Additional information can be obtained including package description, product type name, proprietary name, Drug Enforcement Administration (DEA) schedule, package size, among others.

The information processor can perform various rules in the remote server to identify cartridges that, for example, have been recalled, expired, or previously used. It may also perform security checks such as verifying that the barcode was originally generated from a verified system for generating barcode. The remote server can also generate a digital signature as a further step to ensure the validity of the barcode.

The resulting information is digitized and as such, processes including the preparation and administration of medication, documentation, inventory, and billing, which are usually performed manually, can be performed automatically.

A database client can accept pricing data from Medicaid (https://data.medicaid.gov/) (4) or others. Pricing data can also be enterer manually to give visibility to medication data or calculate inventory terminology including the ones below:

Table 1: Inventory terminology
1. Prescription inventory
2. New prescriptions
3. Renewed prescriptions
4. Average prescription charge
5. Government programs (Medicaid and Medicare Part D)
6. Other third-party programs
7. Percentage of prescriptions dispensed generic
8. Cost of goods sold
9. Prescription sales
10. Inventory turn days
11. Inventory turnover (annual)
12. Inventory turnover days
13. Prescription inventory turnover (annual)
14. Prescription inventory turnover days
15. Prescriptions costs
16. Sales per employee
17. Prescription sales per square foot
18. Sales to assets
19. Return on investment
20. Debt to worth
21. Current ratio
22. Quick ratio
23. Accounts receivable turnover (annual)
24. Accounts receivable collection (days)
25. Accounts payable turnover (annual)
26. Accounts payable turnover (days)
27. Cost of dispensing formula (5)

### Medication is Dispensed from Vials by Open Loop Control

The medication delivery device uses vials as cartridges to dispense medication. Various techniques can be contemplated to dispense medication from a vial including an air-over-fluid technique at a constant flow with the medication delivery device.

Vials are closed cartridges with medication that comprises fluid over a column of air. In an air-over-fluid technique, air is forced into the vial with positive pressure in one port and medication inside is displaced through another port. As medication is dispensed, the volume of air increases as the volume of medication decreases. In other words, the non-compressible fluid in the vial is replaced with compressible air. Because the cartridge is rigid and made of glass or plastic, the cartridge cannot collapse on itself and evacuate air as in the case with a flexible bag. In order to administer a constant flow of fluid over time, the pressure delivered by the pump inside the vial must increase over time.

The medication delivery device uses a constant positive pressure pump at a constant voltage and a pressure controller consisting of a pressure sensor and variable orifice valve. The pressure sensor has a continuous air path with the air within the vial to measure and maintain ambient atmospheric pressure when the device is not administering medication. An air-over-fluid technique using positive pressure is used to administer medication from vials.

When the medication delivery device receives an order to administer medication, the medication delivery device interprets data from the EHR, CPOE, and CDSS to determine flow, which is volume over time. The pump turns on and the pressure controller determines the voltage to apply to the variable orifice valve to allow medication to be administered from the vial constantly. The pressure inside the vial increases to dispense a constant flow of fluid. The orifice of the valve where air is expelled will increase with time to maintain constant pressure inside the vial and constant flow out of the vial.

Changes to temperature and altitude can affect the pressure within a closed cartridge in predictable ways and are governed by Henry's law, where the dissolved air in a fluid is proportional to its partial pressure above the fluid. When a vial is punctured by the administration set has an increase in temperature from being left out in the sun or increases in altitude from a patient being medically evacuated by helicopter in austere clinical environments, the pressure in the cartridge increases. This increase in pressure inside the cartridge may lead to an over infusion of medication from pumps, and air bubbles that may be present in administration set can expand according to Boyle's law. Likewise, a decrease in temperature and altitude, from the medication delivery device descending in an aircraft or submarine, can decrease the pressure in the vial and can cause an under infusion from pumps (6).

These challenges imposed on this medication administration system with the pressure pump and controller are handled in the same fashion as administering medication at room temperature and sea level. Variations in medication administration during changes in temperature and altitude are compensated by the pressure sensor and variable orifice valve.

The medication delivery device is currently designed to minimize variations in medication delivery from changes in temperature and altitude. First, bubbles that form within the vial should float to the top of the vial, which has a column of air. Second, the device has a valve that can vent the vial when medication is not being administered. Third, it has a pressure controller that continuously measures the pressure within the vial and can adjust its orifice and therefore the pressure infused into the vial and medication dispensed from the vial.

### Dispensing of Medication is Verified by Closed Loop Controls

The medication delivery device uses closed loop controls to verify dispensing of medication with camera by artificial intelligence. The technique is to non-invasively identify the starting and change in the fluid level line and volume in a closed cartridge in real-time. Most parenteral medications are clear with the viscosity and appearance of water, and they are often packaged in equally clear vials.

The medication delivery device uses two main methods to verify the volume of fluid administered: fully convolutional neural network (FCN) and machine vision.

The same camera to read the barcode can be used to with closed loop control. The FCN uses an image from the camera to produce a pixelized annotation for semantic segmentation or delineation of fluid from glass (7).

First, based on information from the NDC from the barcode, the medication delivery device can identify the size of the vial. Predictions of the vial outline and fluid level line can be made, and the view of the camera focuses to the expected fluid level line. As the fluid dispenses from the vial, the fluid level line should move and bubbles form from the bottom and move up until the fluid level line in the vial.

Attention can be set to the vial outline and estimated fluid level line for semantic segmentation by FCN. Second, FCN can independently determine the vial outline and set the region of interest (ROI) for semantic segmentation of the fluid level line within the vial. One technique to use with the FCN is to apply a valve filter to focus attention to the known position of the vial in the image. A ROI mask is placed into the FCN in front of a binary map representation of the image. Pixels of the ROI is set to one, and the background set to zero (8).

In addition, machine vision can be used in conjunction with FCN to provide independent information. Any variation of the fluid level line by more than 5% is rejected. Various techniques are used including line object and frame difference. For line object, camera additionally detects angle of the line from the 0 degrees.

With frame difference, the camera looks for changes in the fluid level line once the starting line is determined and calculates the volume of fluid that was dispensed and remaining in the vial.

If there is agreement between each method of line detection, the fluid inside the cartridge is highlighted and a pixelized annotation of the fluid is made. The image is further annotated to identify every 0.1 ml of the fluid. As fluid is dispensed from the vial, a comparison is made between the volume delivered according to the CPOE of the EHR and annotated fluid missing from the vial.

Because the device uses an air-over-fluid technique that forces air into the cartridge to displace fluid, the camera expects to detect bubbles forming within the vial to ensure that the device is dispensing. If the device commands the medication delivery device to dispense and there are no bubbles seen within the vial, the pump is not operating properly and will not administer medication.

The ROI map is a binary image with pixels marking the ROI as 1 and the rest of the image as 0. It is entered into the FCN and superimposed on the image. The image, ROI, and machine vision are separately and independently evaluated by a convolution layer. Image filters convolved with bias addition of the image from the FCN and machine vision generates a feature map, and valve filters of the ROI from the FCN is convoluted similarly generate a relevance map. A normalized feature map is made from the feature and relevance maps. Elements from the maps undergo element wise multiplication are passed through a Rectified Linear Unit (ReLU) to eliminate negative map elements to produce a normalized map that is passed though the next layer of the FCN.

### Prevention of Air Embolism

Once the medication is dispensed from the vial into the administration set, air should not enter the administration set to reduce the risk of air embolism to the patient. Air can be kept separate from the fluid in a variety of ways. First, one camera is positioned in front of each vial such that bubbles must be seen in the vial for the device to be administering medication. Second, the air orifice is placed above the medication orifice in the administration set such that medication can continue to drain from the vial beyond the point that the camera can see bubbles forming in the vial, and when bubbles are not seen in the vial, the device turns the pump off. Third, the medical device incorporates a mechanical stabilizer that does not use power to decrease low- and high-frequency vibrations to keep the vials upright so that air is not entailed into the fluid path of the administration set. Fourth, the microprocessor incorporates an accelerometer to measure changes in velocity and gyroscope to calculate the degree of the X-, Y-, and Z- axes to report the level of the medication delivery device.

### SUMMARY

The disclosure provides for a medication delivery device. The disclosure further provides that the medication delivery device can deliver metered doses of medication in an accurate and precise manner.

The disclosure provides for a medication delivery device delivering metered doses of fluids in an accurate and precise manner comprising: (a) an elongate member having a first and second end; (b) a plurality of fluid cartridge adapters spaced along the length of the elongate member, wherein each adapter is adapted to receive fluid from a removable fluid cartridge; (c) a fluid channel that is centrally disposed within the elongate member and fluidly linked to the fluid cartridge adapters, wherein the fluid channel's first end is enclosed in the elongate member while the second end is fluidly linked to an exit portal; (d) an external computer that programs the medication delivery device at the time of order entry; (e) one or more pressure modifying devices that are pressureably linked to the fluid channel so that a change in pressure from the pressure modifying device causes fluid to move into the fluid channel; (f) one or more valves to prevent fluid from moving back into cartridge and to ensure fluid only moves to exit portal; (g) one or more scanners to detect one or more machine-readable indicators on a removable fluid cartridge once the removable fluid cartridge is inserted into the medication delivery device, and then the scanners can output information provided by the machine-readable indicator to a computer; and (h) one or more scanners to detect the volume of the fluid in the cartridge.

In an embodiment, the disclosure provides for a medication delivery device disclosed herein comprising a computer that has an attached or wireless device to communicate with another computer to receive demographic, laboratory, prescription, or entire electronic health record data from patients. In another embodiment, the medication delivery device receives data to semi autonomously or autonomously program the administration of fluid from the medication delivery device. In yet another embodiment, the medication delivery device modifies the program based on interpretation of the data.

In a particular embodiment, the disclosure provides for a medication delivery device disclosed herein comprising a plurality of pre-filled removable fluid cartridges, wherein each removable fluid cartridge comprises a fluid ejection port, an injection port, a machine-readable indicator, and a window to view the inside volume of the cartridge.

In an embodiment, the disclosure provides that one or more barcode reader comprising a camera, sensor, scanner, or scanner that reads the barcode from the cartridge. In another embodiment an operatively connected information processor can be used to read the barcode to accurately and quickly identify characteristics of the medication.

In one particular embodiment of this patent, an information processor receives a machine-readable indicator or plurality of machine-readable indicators on cartridges and consists of an information processor operatively connected to a computer to interpret the machine-readable indicator information and output information provided by such indicator to an operatively connected locally to a computer and a remote server.

In another particular embodiment of this patent, the output information generated by the medication delivery device for generating and using barcode can be used to generate reports on inventory and supply chain management, reordering, usage, billing, among other reasons.

In a particular embodiment of this patent, the scanner records the time, date, and location onto a remote server to track current inventory on hand of medications throughout a health care facility and is visible though creating reports. This system is designed to support supply chain management and just-in-time (JIT) inventory strategy.

In another embodiment of this patent, the scanner can be used to generate reports to assign charges to patients or expense to related parties.

In another embodiment of this patent, the scanner receives the credentials of the user who may be a pharmacist, a pharmacist technician, or other qualified personnel to handle cartridges.

In an embodiment, the disclosure provides for a medication delivery device disclosed herein comprising a plurality of fluid cartridge holders physically attached to a turning motor to facilitate the presentation of the cartridge to a machine-readable indicator or sensor.

In a particular embodiment, the disclosure provides for the medication delivery device disclosed herein comprising an attached portion of the structural housing can slide freely vertically up and down or horizontally side to side to further prevent movement of elongate member. In one embodiment, one or more pegs fitting into one or more corresponding holes in the elongate member to restrict the elongate member's movement. In another embodiment, the pegs are pressureably in contact and connected with pressure modifying device and pressure controller and centrally disposed in elongate member. In a further embodiment, the structural housing is further comprised of tubing, wherein the tubing cannot be reversibly attached to the pegs. In yet a further embodiment one or more injection ports of an elongate member's reversibly attached to the removable fluid cartridges.

In a further embodiment, the pump is controllable by a computer or software such as a CPOE, EHR, or database semi autonomously or autonomously. In yet a further embodiment, the pump's operation can be controlled by commands entered by a user on user interface connected to a computer. In a certain embodiment, a user interface may be connected to a computer that can be used to control the pump either directly, wirelessly, or remotely. In a further embodiment, the pump may be controlled by a computer that is accessed through the Internet, network, or phone line from commands entered on a user interface that is connected to an alternate computer.

In a particular embodiment, the disclosure provides for a medication delivery device disclosed herein comprising a computer. In another embodiment, the computer can receive and display the output from the scanner. In a further embodiment, the one or more information storage devices provide information, including, but not limited to, EHR and CPOE, and contraindications for therapeutic agents. In yet a further embodiment, the user interface can relay information to the user from the computer or from connected information storage devices, including allergies, warnings, contraindications, DDI, organ failure, patient laboratory data, patient vital signs, patient measured data, dosing recommendations, and patient specific concerns. In another embodiment, a computer comprising a user interface can integrate one or more global positioning system location sensors in the medication delivery device, elongate member, or cartridges for inventory management.

In a particular embodiment, the disclosure provides for a fluid flow meter disclosed herein comprising a camera, sensor, or scanner to verify the starting, operating, and ending volume of the cartridge. In another embodiment, the fluid flow meter uses FCN, machine vision, computer vision, or algorithms to determine the volume in the cartridge. In yet another embodiment, one camera determines the fluid level line of a plurality of cartridges with a wide-angle lens to bring the image in closer view. In another embodiment, one camera determines the fluid level line of a plurality of cartridges with one or a plurality of mirrors. In yet another embodiment, one camera determines the fluid level line of a plurality of cartridges with one or a plurality of fiber optic pipes. In another embodiment, other techniques can be used to determine the fluid level line including ultrasonic means or mechanical flow meter.

A method for controlling use of the device, comprising: (1) prompting a user to enter information about personnel; (2) retrieving patient information; (3) detecting a machined readable indicator on the surface of the removable fluid cartridge by a scanner when the cartridge is inserted into the device; (4) outputting information from the scanner to an attached computer about the information provided by the machine-readable indicator; (5) using an algorithm that is programmed into the computer to calculate a proper dose for administering to the patient based on the information outputted to the computer by scanner; (6) reading the fluid level line and measuring the fluid volume inside the cartridge; and (7) activating a pressure modifying device and pressure controller to apply pressure to the removable fluid cartridge so as to force fluid from the removable fluid cartridge until the calculated dosage is achieved.

### BRIEF DESCRIPTION OF THE DRAWINGS

FIG. 1 is a top-down angled front view of a medication delivery device showing certain features, such as an elongate member, a plurality of cartridges, a touchscreen console, structural housing can slide freely vertically up and down or horizontally side to side, speaker, and power switch.

FIG. 2 is a top-down angled top exploded view of a medication delivery device.

FIG. 3 is a back cut away view of a tubed-shaped elongate member 105 with a plurality of cartridges 110 physically attached to a plurality of raised fluid cartridge adapters 305 to show a fluid channel 310 and pressure channel 315 within the elongate member 105, two one-way valves per channel, and hole 410 pressureably connected to the fluid cartridge adapters through pressure channel 315.

FIG. 4 is a top-down angled exploded back view of an elongate member with a plurality of cartridges to show two one-way valves per channel and hole.

More specifically, the disclosure provides for a medication delivery device that can accept a plurality of pre-filled removable fluid cartridges, wherein the device can then dispense fluid from one or more of these cartridges in a controlled manner by using a pressure modifying device, such as a pump. Moreover, a medication delivery device disclosed herein can detect when a fluid cartridge is inserted in the device, and identify the contents.

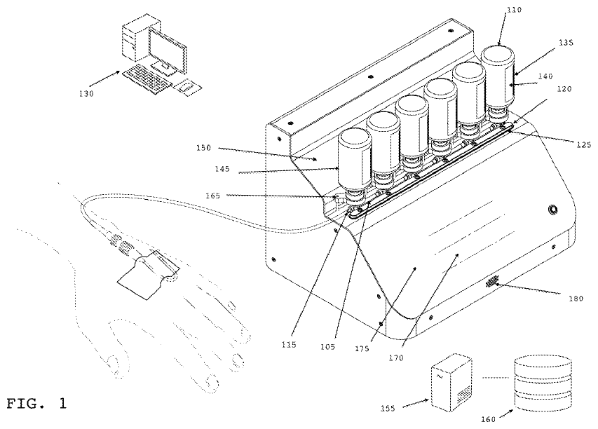

An overview of an exemplary drug delivery device of the disclosure is presented in FIG. 1. An elongate member 105 is comprised of a centrally disposed fluid channel (not shown) and one or more fluid cartridge adapters (not shown), such that fluid channel is fluidly connected to the fluid cartridge adapters through the fluid channel. The elongate member 105 is also comprised of a centrally disposed pressure channel (not shown) and one or more fluid cartridge adapters, such that pressure channel is pressureably connected to the fluid cartridge adapters through the cartridge adapter. The fluid cartridge adapters are adapted to receive fluid from removable fluid cartridges 110, such that fluid can be forced or drawn from the removable fluid cartridges 110 by using one or more pressure modifying devices (not shown) and pressure controllers (not shown). The fluid can then travel down the fluid channel an out an exit portal 115. In a certain embodiment, the medication delivery device may further comprise a structural housing 120 that is dimensioned so as to contact an elongate member 105 in order to restrict the movement of the elongate member and to provide overall stability to the device. In particular, structural housing 120 can slide freely vertically up and down or horizontally side to side to further prevent movement of elongate member 105. In the preferred embodiment, one or more pegs (not shown) fitting into one or more corresponding holes (not shown) in the elongate member 105 to restrict the elongate member's 5 movement. In another embodiment, the pegs are pressureably in contact and connected with pressure modifying device and pressure controller and centrally disposed in elongate member 105. In a further embodiment, the structural housing 120 is further comprised of tubing, wherein the tubing cannot be reversibly attached to the pegs. In yet a further embodiment one or more injection ports of an elongate member's 5 reversibly attached to the removable fluid cartridges 110. In another embodiment, an elongate member 105 comprising a fluid channel, has a plurality of raised hollow projections, such as spikes, nozzles, or tips, that are so dimensioned so that the elongate member 105 hollow projections can slidably insert into the cartridge 110, such that cartridge 110 is fluidly connected to fluid channel.

In a particular embodiment, the disclosure provides for removable fluid cartridge 110 further comprising a pierceable port (not shown). In a certain embodiment, the pierceable port is located on one of the accessible faces of the removable fluid cartridge 110. Generally, the pierceable port should be located so as to facilitate access to the fluid in the removable fluid cartridge 110 for the purposes of mixing, withdrawing, and or injecting a gas to the fluids inside. The pierceable port is covered with a material, such as foil, plastic, or a membrane, that can be punctured by a solid object, such as a needle.

The removable fluid cartridge comprises a lighted display 125, wherein the lighted display indicates the status of the cartridge 110.

The medication delivery device is programmed by physicians or other health care personnel entering a medication order into the CPOE, EHR, or CDSS with a computer 130.

A scanner (not shown) is a machine capable of detecting and identifying a machine-readable indicator 135 or plurality of machine-readable indicators 135 on a label 140. Window 145 of cartridge 110 is shown between label 140. A scanner can include, but is not limited to, barcode readers, cameras, radio frequency receivers, scanners, or a combination thereof. Barcode readers, cameras, radio frequency receivers, scanners, or a combination thereof of scanner interprets the machine-readable indicator 135 and transmits this information connected wirelessly, such as by Bluetooth, router, or modem; or remotely over a network, such as through a LAN or through the Internet.

The scanner also uses FCN, machine vision, computer vision, or algorithms to read the fluid level line and volume inside the cartridge 110 from the window 145.

A wide-angle lens 150 attached to scanner bring the image in closer view. The information processor can perform various rules in the remote server to identify cartridges that, for example, have been recalled, expired, or previously used. It may also perform security checks such as verifying that the barcode was originally generated from a verified system for generating barcode. The remote server can also generate a digital signature as a further step to ensure the validity of the barcode.

A scanner for reading information of interest including barcodes (barcode reader) and human-readable information [optical character recognition (OCR)] and operatively connected to a medication delivery device. A medication delivery device has a suitable wireless connection such as a Bluetooth, Wi-Fi, ZigBee, among others to an information storage device 155. A medication delivery device is also operatively connected to a web console network such as by a cable; connected wirelessly, such as by Bluetooth, router, or modem; or remotely over a network, such as through a LAN or through the Internet.

An information storage device 155 can store and may also retrieve information and includes hard drives, tapes, flash drives, computers, and servers. An information storage device 155 may be directly connected to a database 160 like a computer, by a cable or plug; connected wirelessly, such as by Bluetooth, router, or modem; or remotely over a network, such as through a LAN. Data stored in database 160 can be accessed via Bluetooth, router, or modem; or remotely over a network, such as through a LAN.

Fluid cartridge holder 165 may be comprised of any material or any combination of materials. In a certain embodiment fluid cartridge holder 165 is comprised of a magnet that placed above an electromagnet (not shown). The fluid cartridge holder 165 can be of any shape, as long as a portion of a removable fluid cartridge 110 can be placed inside the fluid cartridge holder 165. In the preferred embodiment, the fluid cartridge holder 165 is circular. In an alternative embodiment, the structural housing 120 can be dimensioned to receive a removable fluid cartridge 110. In yet another alternative embodiment, the disclosure provides that the medication delivery device does not comprise a fluid cartridge holder 165.

The fluid cartridge holders 165 have an L shaped hingeable door (not shown) is normally open and closes when cartridge is placed into fluid cartridge holders 165. Underneath fluid cartridge holders 165, an electromagnetic can be turned on to lock L shaped hingeable door and turn the fluid cartridge holders 165 to facilitate the presentation of the cartridge to a camera to read the machine-readable indicator 135 and fluid level line and volume. The polarity of the electromagnetic can be reversed to allow similar polarity of the electromagnetic to be presented to the fluid cartridge holders 165 and pull the fluid cartridge holders 165 and cartridges 110 onto the fluid cartridge adapter.

When the electromagnetic is be turned off, the cartridge 110 can be removed by pulling up on it and the L shaped hingeable door opens. In another embodiment, the fluid cartridge holders 165 can also turn the attached to a turning motor to facilitate the presentation of the cartridge to a machine-readable indicator 135. In another embodiment, a solenoid is engaged to the neck of the cartridge 110 to secure the cartridge 110 to prevent tampering and theft and another solenoid can pull the fluid cartridge holders 165 and cartridges 110 onto the fluid cartridge adapter.

In a certain embodiment, a fluid cartridge holder 165 is dimensioned so that it can be attached to the medication delivery device. In another embodiment, the fluid cartridge holder 165 is near to fluid cartridge adapter 305. In another embodiment, a fluid cartridge holder 165 is dimensioned so that it can be attached to structural housing 120. In yet another embodiment, a fluid cartridge holder 165 is dimensioned so that it can be attached to elongate member 105. The attachable fluid cartridge holder 165 can be attached or near to a fluid cartridge adapter 305, structural housing 120, and/or elongate member 105, by any manner known in the art. In a certain embodiment, the disclosure provides that the fluid cartridge holder 165 can be irreversibly attached to a fluid cartridge adapter 305, structural housing 120, and/or elongate member 105. Examples of irreversible attachment include, but are not limited to, welding, molding, cementing, gluing, and riveting. In an alternative embodiment, the disclosure provides that a fluid cartridge holder 165 can be reversibly attached to a fluid cartridge adapter 305, structural housing 120, and/or elongate member 105. Examples of reversible attachment include, but are not limited to, locking pins; threaded connections, such as being able to screw a holder 165 onto an adapter; screws; lock and key type connections; retaining rings; and clasps. An exemplary process of attaching a fluid cartridge holder 165 to structural housing 120 by using a solenoid is presented. A fluid cartridge holder 165 is placed on a solenoid that can move freely vertically up and down or horizontally side to side in a piston-like motion.

A touchscreen 170 is user console to input or display information. The touchscreen 170 displays vital information required for user-control of the system including patient names, ID numbers, patient medical history, patient vital statistics, patient weight, drug indications, medication names and other relevant information which can be retrieved by the medication delivery device. The touchscreen 170 also displays visual indication of battery life and status of wireless communication. A reader 180 is used to scan the credentials of the user who may be a pharmacist, a pharmacist technician, nurse, physician, or other qualified personnel to handle cartridges. The speaker 180 enunciates information provided on the machine-readable indicator of one or more removable fluid cartridges after the removable fluid cartridges are inserted into a medication delivery device disclosed herein. The turns the device on and off.

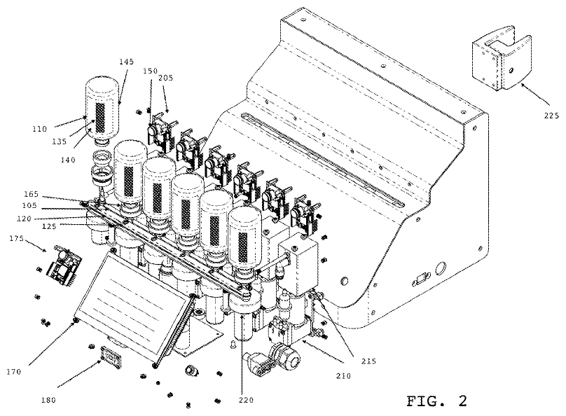

FIG. 2 is a top-down angled top exploded view of a medication delivery device. A scanner 205 is a machine capable of detecting and identifying a machine-readable indicator 135 or plurality of machine-readable indicators 135 on a label 140. A scanner 205 can include, but is not limited to, barcode readers, cameras, radio frequency receivers, scanners, or a combination thereof. Barcode readers, cameras, radio frequency receivers, scanners, or a combination thereof of scanner interprets the machine-readable indicator 135 and transmits this information connected wirelessly, such as by Bluetooth, router, or modem; or remotely over a network, such as through a LAN or through the Internet.

The scanner 205 also uses FCN, machine vision, computer vision, or algorithms to read the fluid level line and volume inside the cartridge 110.

A wide-angle lens 150 attached to scanner bring the image in closer view. The information processor can perform various rules in the remote server to identify cartridges that, for example, have been recalled, expired, or previously used. It may also perform security checks such as verifying that the barcode was originally generated from a verified system for generating barcode. The remote server can also generate a digital signature as a further step to ensure the validity of the barcode.

One or more pressure modifying devices 210, such as diaphragm pumps, that can exert positive pressure on the fluid contained in the cartridges 110; one or more pressure controllers 215, including a pressure sensor and a variable orifice valve in each, that can control positive pressure on the fluid contained in the cartridges 110; one or more scanners 205, such as a camera, located behind each fluid cartridge adapter and positioned to read the machine-readable indicator 135, such as a bar code, present on the side or bottom face of the cartridge 110 once a cartridge 110 is inserted into a fluid cartridge adapter, wherein the scanner can then output information provided on the machine-readable indicator 135 to an information storage device 155; one or more scanners 205, such as a camera, located behind each fluid cartridge adapter and positioned to read the cartridge fluid level line and volume real-time once a cartridge 110 is inserted into a fluid cartridge adapter, wherein the scanner 205 can then output information provided on the machine-readable indicator 135 to an information storage device 155; an information storage device 155 that can receive the output from the scanner 205, such as a camera, and then present information related to the scanner's 205 output on a touchscreen 170, wherein the information storage device 155 may also be connected directly, wirelessly, or remotely to one or more pressure modifying devices, and/or one way valves to control the flow of pressure each removable fluid cartridge 110 receives, control the amount of fluid that is released in the fluid channel, control the amount of fluid that is released into the exit portal, and/or be connected to and can present information from one or more information storage devices 150; and/or an information storage device 155 that can receive input from user defined commands from an external information storage device 155 or software and then present information related to the scanner's 205 output on a display, wherein the information storage device 155 may also be connected directly, wirelessly, or remotely to one or more pressure modifying devices 210 and one or more pressure controllers 215 to control the flow of pressure each removable fluid cartridge 110 receives, control the amount of fluid that is released in the fluid channel, control the amount of fluid that is released into the exit portal, and/or connect to and can present information from one or more information storage devices 150; one or more scanners located behind each fluid cartridge adapter and positioned to read the cartridge fluid level line and volume once a removable fluid cartridge 110 is inserted into a fluid cartridge adapter, wherein the scanner 205 can then output information provided from the fluid cartridge 110 to an information storage device 155.

Electromagnet 220 is shown underneath fluid cartridge holders 165 and turns the fluid cartridge holders 165, locks the L shaped hingeable door, and pulls the fluid cartridge holders 165 and cartridges 110 onto the fluid cartridge adapter 305.

Clamp 225 is attached to the back of the medication delivery device to attach to a pole to stabilize the device.

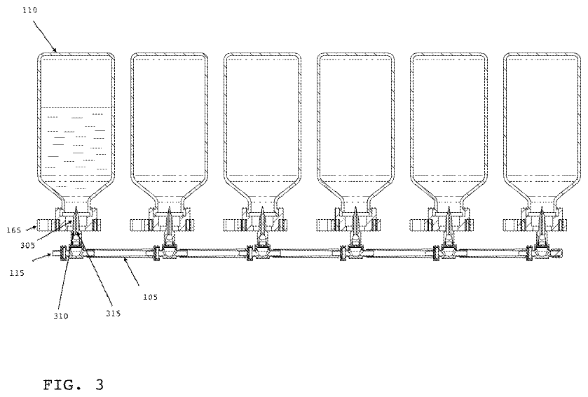

FIG. 3 is a back cut away view of a tubed-shaped elongate member with a plurality of cartridges physically attached to a plurality of raised fluid cartridge adapter to show a fluid and pressure channel within the elongate member, two one-way valves per channel, and hole pressureably connected to the fluid cartridge adapters through pressure channel.

Elongate member 105 has a plurality of cartridges 110 physically attached to a plurality of raised fluid cartridge adapter 305 to allow fluid to access a fluid channel 310 within the elongate member 105 and pressure channel 315 to access the inside of the cartridges and two one-way valves 405 per channel. Elongate member 105 has a first end and a second end. Elongate member 105 may be comprised of any material or any combination of materials. In a certain embodiment the elongate member 105 is comprised of plastic and silicone. In a further embodiment the elongate member 105 is comprised of rubber. In yet another embodiment, the elongate member 105 is comprised of material that is inert to fluids. In yet a further embodiment, the elongate member 105 is comprised of metal. Generally, elongate member 105 should be of a sufficient length so that it can potentially accommodate one or more removable fluid cartridges 110. In a particular embodiment, elongate member 105 is from 2 to 36 inches in length. In another embodiment, elongate member 105 is from 2 to 24 inches in length. In yet another embodiment, elongate member 105 is from 6 to 24 inches in length. In a further embodiment, elongate member 105 is from 10 to 24 inches in length. Elongate member 105 comprises at least one wall defining an enclosed space or tube. Moreover, the width of elongate member 105 can vary. The first and second end of elongate member 105 may have relatively the same dimensions or alternatively the ends may have different dimensions. While the figures in this application present an elongate member 105, as being substantially tube-shaped, the disclosure provides for elongate member 105 having substantially a non-tubed shape, a T shape, or a non-T shape. Any shape for elongate member 105 is acceptable, so long as the elongate member 105 can further comprise a fluid channel and one or more fluid cartridge adapters 305 and a pressure channel. In a certain embodiment, elongate member 105 has a substantially flat lower wall so as to provide stability when elongate member 105 is placed on a flat surface. However, elongate member 105 may be in contact with a structural housing 120, such that structural housing 120 can provide needed structural stability. In such a case, elongate member 105 can have a bottom face that is substantially not flat or have portions that are substantially flat and not flat.

Elongate member 105 comprises a fluid channel 310, pressure channel 315, and a plurality of fluid cartridge adapter 305. Elongate member 105 has a first end and a second end. Elongate member 105 may be comprised of any material or any combination of materials. In a certain embodiment the elongate member 105 is comprised of plastic and silicone. In a further embodiment the elongate member 105 is comprised of rubber. In yet another embodiment, the elongate member 105 is comprised of material that is inert to fluids. In yet a further embodiment, the elongate member 105 is comprised of metal. Generally, elongate member 105 should be of a sufficient length so that it can potentially accommodate one or more removable fluid cartridges. In a particular embodiment, elongate member 105 is from 2 to 36 inches in length. In another embodiment, elongate member 105 is from 2 to 24 inches in length. In yet another embodiment, elongate member 105 is from 6 to 24 inches in length. In a further embodiment, elongate member 105 is from 10 to 24 inches in length. Elongate member 105 comprises at least one wall defining an enclosed space or tube. Moreover, the width of elongate member 105 can vary. The first and second end of elongate member 105 may have relatively the same dimensions or alternatively the ends may have different dimensions. While the Figures in this application present an elongate member 105, as being substantially tube-shaped, the disclosure provides for elongate member 105 having substantially a non-tubed shape, a T shape, or a non-T shape. Any shape for elongate member 105 is acceptable, so long as the elongate member can further comprise a fluid channel and one or more fluid cartridge adapters 305 and a pressure channel. In a certain embodiment, elongate member 105 has a substantially flat lower wall so as to provide stability when elongate member 105 is placed on a flat surface. However, elongate member 105 may be in contact with a structural housing 120, such that structural housing 120 can provide needed structural stability. In such a case, elongate member 105 can have a bottom face that is substantially not flat or have portions that are substantially flat and not flat.

Fluid channel 310 can be any shape. Fluid channel 310 can be centrally disposed in elongate member 105 or alternatively be disposed off-center in elongate member 105. The diameter or cross section of fluid channel 310 can be generally sized to fit the needs of a particular application. For example, for medication applications the fluid channel 310 may need to be of a sufficient size to allow a large volume of one or more fluids to pass down the channel and out the exit portal. Alternatively, for medical applications the fluid channel 310 may require a smaller diameter to account for a low volume of fluid passing into the fluid channel 310. Fluid channel 310 has at least one opening present in one end of elongate member 105 that is connected to an exit portal. Fluid channel 310, however, in a certain embodiment, can allow for openings being present in both ends of elongate member 105. This second opening could facilitate attaching one or more pressure modifying devices 210, such as pumps, compressors, or gas cylinders, and pressure controller 215 in order to push fluids remaining in fluid channel 310 out into the exit portal 115. This second opening could be also used to attach a reservoir of wash solution so that fluid channel 310 can be cleaned of any residual fluid originating from one or more removable fluid cartridges 110.

In a particular embodiment a fluid cartridge holder 165 is attached to structural housing 120 and not attached to a fluid cartridge adapter 305. In a further embodiment, a fluid cartridge holder 165 is attached to structural housing 120 and attached to a fluid cartridge adapter 305. Generally, the fluid cartridge adapter 305 is adapted to receive a removable fluid cartridge 110 even when a fluid cartridge holder 165 is attached to a fluid cartridge adapter 305. In a certain embodiment, a fluid cartridge adapter 305 comprises a male type receptor that can fit into a matching female type receptor on a removable fluid cartridge 110. In an alternate embodiment, a removable fluid cartridge 110 comprises a female type receptor 35 that can fit into a matching male type nozzle on a fluid cartridge adapter 305.

In a particular embodiment, removable fluid cartridge 110 may further comprise machine-readable indicator 135, so that once removable fluid cartridge 110 is slidably inserted into a fluid cartridge holder 165, machine-readable indicator 135 can be read by scanner (not shown). Scanner can also read fluid level and volume through the window 145. In a further embodiment, machine-readable indicator 135 is orientated to be contiguous with window 145 by slidably inserting removable cartridge 110 into cartridge holder 165 so that notch projection 22 is guided into the matching notch depression.

In a certain embodiment, fluid cartridge 110 may further comprise window 145 between label 140 can be visually seen by a user and/or can be identified by a scanner, when a removable cartridge 110 is inserted into fluid cartridge holder 165.

A fluid cartridge adapter 305 is fluidly connected to fluid channel through a cartridge adapter. Cartridge adapter can be of any length and of any diameter, so long as fluid can flow from a removable fluid cartridge 110 and through cartridge adapter. In a certain embodiment, cartridge adapter may further comprise a one-way valve, so that fluid can only flow from a removable fluid cartridge into fluid channel. In another embodiment, cartridge adapter may further comprise a one-way valve so that once the valve is closed no fluid can flow from the removable fluid cartridge into a fluid channel. Such locking and one-way valves can be either manually, magnetically, and/or electronically opened or closed. Moreover, the disclosure provides embodiments, for the opening or closing of such valves by entering user defined commands on a user interface that is connected to a computer, which is connected to a medication delivery device disclosed herein.

A fluid cartridge adapter 305 can have any shape as long as the fluid cartridge adapter can receive fluid from a removable fluid cartridge 110. In a certain embodiment, a fluid cartridge adapter 305 is a hole in the elongate member 105 that is fluidly connected to fluid channel. A fluid cartridge adapter 305 may be comprised of any material or any combination of materials. In yet another embodiment, the cartridge adapter 305 is comprised of material that is inert to fluids. In a certain embodiment, a fluid cartridge adapter 305 is comprised of plastic. In a further embodiment, a fluid cartridge holder 165 is comprised of rubber. In yet a further embodiment, fluid cartridge holder 165 is comprised of metal. A fluid cartridge adapter 305 can be attached to an elongate member 105 by any means. In alternative embodiment, a fluid cartridge adapter 305 can be reversibly attached to an elongate member 105. For example, a threaded fluid cartridge adapter 305 can be screwed into a matching threaded elongate member 105. In a preferred embodiment, fluid cartridge adapter 305 can be fitted onto a raised hollow portion, such as a nozzle or tip, on elongate member 105. In another embodiment, a fluid cartridge adapter 305 is irreversibly attached to an elongate member 105. Examples of irreversible attachment include, but are not limited to, welding, molding, cementing, gluing, and riveting.

In the preferred embodiment, the diameter or cross section of the pressure channel can be generally sized to fit the needs of a particular application. For example, for medication applications the pressure channel may need to be of a sufficient size to allow a large volume of one or more gas to pass from each of the fluid cartridge adapter to the fluid cartridge by one or more pressure modifying devices, such as pumps, compressors, or gas cylinders and one or more pressure controllers in order to push fluids from the fluid cartridges 15 out into the exit portal.

In another embodiment, fluid channel can be comprised of one or more tubes that are fluidly connected to one or more removable fluid cartridges 110. In a particular embodiment, each fluid cartridge adapter 305 comprises individual tubing that runs down the length of fluid channel and out to the exit portal. Examples of such tubing, including surgical tubing, or pharmaceutical grade tubing. Moreover, the tubing should have a relatively small diameter so that a plurality of tubing can fit within fluid channel. The plurality of tubing may then be combined into a single tube in the exit portal, if so needed, or alternatively remain separate.

An elongate member 105 is comprised of a centrally disposed fluid channel (not shown) and one or more fluid cartridge adapters (not shown), such that fluid channel is fluidly connected to the fluid cartridge adapters through the fluid channel. The elongate member 105 is also comprised of a centrally disposed pressure channel (not shown) and one or more fluid cartridge adapters, such that pressure channel is pressureably connected to the fluid cartridge adapters. The fluid cartridge adapters are adapted to receive fluid from removable fluid cartridges 110, such that fluid can be forced or drawn from the removable fluid cartridges 110 by using one or more pressure modifying devices (not shown) and pressure controllers (not shown). The fluid can then travel down the fluid channel an out an exit portal 115. In a certain embodiment, the medication delivery device may further comprise a structural housing 120 that is dimensioned so as to contact an elongate member 105 in order to restrict the movement of the elongate member and to provide overall stability to the device. In particular, structural housing 120 can slide freely vertically up and down or horizontally side to side to further prevent movement of elongate member 105. In the preferred embodiment, one or more pegs fitting into one or more corresponding holes in the elongate member 105 to restrict the elongate member's 5 movement. In another embodiment, the pegs are pressureably in contact and connected with pressure modifying device and pressure controller and centrally disposed in elongate member 105.

The elongate member 105 comprises a plurality, such as six, fluid cartridge adapters 305, wherein each fluid cartridge adapter 305 further comprises two one-way valves 405 which prevents fluid from flowing into a removable fluid cartridge 110 from the fluid channel 310 and flowing away from the patient, syringe, or similar device in the fluid channel 310; a fluid channel 310 that is enclosed within the elongate member 105 and in fluidly contact with each fluid cartridge adapter 305, and where one end of the fluid channel is enclosed in the elongate member 105 and the other end is connected to an exit portal 115; a pressure channel 315 that is enclosed within the elongate member 105 and in pressureably contact with each fluid cartridge adapter 305; one or more removable pre-filled cartridges 110 further comprising a machine-readable indicator 135 located on the side or bottom face of the cartridge 110.

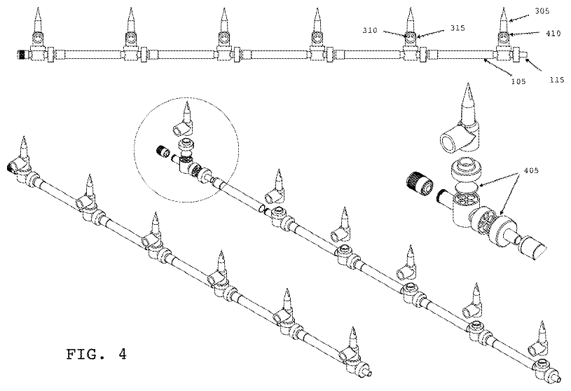

FIG. 4 is a multi-view of an elongate member 105 to show two one-way valves 405 per channel, hole 410, and fluid channel and pressure channel.

In one embodiment, one or more pegs fitting into one or more corresponding holes 410 in the elongate member 105 to restrict the elongate member's 105 movement. In another embodiment, the pegs are pressureably in contact and connected with pressure modifying device 210 and pressure controller 215 and centrally disposed in elongate member.

A user selects that the fluid is to be administered using a user entered command on the user interface, wherein the computer then calculates the dose based on the information outputted by the scanner and the information inputted about the patient, wherein the computer adjusts the dose if the user inputted that the patient has organ failure, contraindication, or other concerns by the computer, medication delivery device, CDSS, or other system. Upon confirming the dose by the user, the dispensing system is activated and one or more pressure modifying devices that can exert positive pressure on the fluid contained in a removable fluid cartridge and one or more one or more pressure modifying devices and one or more pressure controllers exerts positive pressure in the removable fluid cartridge so as to force fluid from the removable fluid cartridge out the exit portal and of elongate member, wherein the fluid is expelled out the fluid channel to the exit portal where the fluid is administered to the patient, syringe, or similar device. One or more one or more pressure modifying devices and one or more pressure controllers continues to exert positive pressure on the removable fluid cartridge until the calculated dosage is reached, at which the dispensing system terminates.

In an exemplary medication delivery device of the disclosure, the medication delivery device is comprised of an elongate member comprising a plurality, such as six, fluid cartridge adapters, wherein each fluid cartridge adapter further comprises two one-way valves which prevents fluid from flowing into a removable fluid cartridge from the fluid channel and flowing away from the patient, syringe, or similar device in the fluid channel; a fluid channel that is enclosed within the elongate member and in fluidly contact with each fluid cartridge adapter, and where one end of the fluid channel is enclosed in the elongate member and the other end is connected to an exit portal; a pressure channel that is enclosed within the elongate member and in pressureably contact with each fluid cartridge adapter; one or more removable pre-filled cartridges further comprising a machine-readable indicator located on the side or bottom face of the cartridge; one or more pumps, such as diaphragm pumps, that can exert positive pressure on the fluid contained in the cartridges; one or more pressure controllers, including a pressure sensor and a variable orifice valve in each, that can control positive pressure on the fluid contained in the cartridges; one or more scanners, such as a camera, located behind each fluid cartridge adapter and positioned to read the machine-readable indicator, such as a bar code, present on the side or bottom face of the cartridge once a cartridge is inserted into a fluid cartridge adapter, wherein the scanner can then output information provided on the machine-readable indicator to a computer; one or more scanners, such as a camera, located behind each fluid cartridge adapter and positioned to read the cartridge fluid level line and volume real-time once a cartridge is inserted into a fluid cartridge adapter, wherein the scanner can then output information provided on the machine-readable indicator to a computer; a computer that can receive the output from the scanner, such as a camera, and then present information related to the scanner's output on a display, wherein the computer may also be connected directly, wirelessly, or remotely to one or more pressure modifying devices, and/or one way valves to control the flow of pressure each removable fluid cartridge receives, control the amount of fluid that is released in the fluid channel, control the amount of fluid that is released into the exit portal, and/or be connected to and can present information from one or more information storage devices; and/or a computer that can receive input from user defined commands from an external computer or software and then present information related to the scanner's output on a display, wherein the computer may also be connected directly, wirelessly, or remotely to one or more pressure modifying devices to control the flow of pressure each removable fluid cartridge receives, control the amount of fluid that is released in the fluid channel, control the amount of fluid that is released into the exit portal, and/or connect to and can present information from one or more information storage devices; one or more scanners located behind each fluid cartridge adapter and positioned to read the cartridge fluid level line and volume once a removable fluid cartridge is inserted into a fluid cartridge adapter, wherein the scanner can then output information provided from the fluid cartridge to a computer.

In a further embodiment, a plurality of fluid cartridge adapters consisting of a hole that have a centrally orientated hollow post and side walls in pressureably contact with a pressure channel that is enclosed within the elongate member and in pressureably contact with each fluid cartridge adapter, such that a small bore connector for gases (ISO/DIS 80369-2: Small bore connectors for liquids and gases in healthcare applications—Part 2: Connectors for breathing systems and driving gases applications), luer connector, or similar structure for healthcare applications of the hole of the fluid cartridge adapters of the elongate member can slidably insert into the small bore connector, and wherein once the cartridge is inserted into the hole of the fluid cartridge adapter it will remain in a substantially upright position.

The disclosure provides for a medication delivery device comprising one or more pressure modifying devices, such as diaphragm pumps, that can exert positive pressure on the fluid contained in the cartridges; one or more pressure controllers, including a pressure sensor and a variable orifice valve in each, that can control positive pressure on the fluid contained in the cartridges, that can be controlled by an external computer or software from orders placed by health care personnel or database such as a CPOE and EHR then present information related to the scanner's output on a display. The medication delivery device of the disclosure can therefore automate several tasks that are normally performed manually by health care personnel.

### REFERENCES

1. Fraind D B, Slagle J M, Tubbesing V A, Hughes S A, Weinger M B. Reengineering Intravenous Drug and Fluid Administration Processes in the Operating Room. Anesthesiology [Internet]. 2002 Jul [cited 2019 Sep 3];97(1):139-47. Available from: http://www.ncbi.nlm.nih.gov/pubmed/12131115
2. Kumar V, Barcellos W A, Mehta M P, Carter J G. An analysis of critical incidents in a teaching department for quality assurance. A survey of mishaps during anaesthesia. Anaesthesia [Internet]. 1988 Oct [cited 2018 Nov 21];43(10):879-83. Available from: http://www.ncbi.nlm.nih.gov/pubmed/3202301
3. Adapa R M, Mani V, Murray L J, Degnan B A, Ercole A, Cadman B, et al. Errors during the preparation of drug infusions: a randomized controlled trial. Br J Anaesth [Internet]. 2012 Nov [cited 2019 Oct 13];109(5):729-34. Available from: http://www.ncbi.nlm.nih.gov/pubmed/22850220
4. Data.Medicaid.gov [Internet]. [cited 2020 Jan 23]. Available from: https://data.medicaid.gov/
5. Diab S, Guinand M. NCPA Creative/Design Director NCPA Digest Sponsored By Cardinal Health [Internet]. 2013 [cited 2020 Jan 23]. Available from: www.ncpanet.org.
6. Siebert J N, Ehrler F, Lovis C, Combescure C, Haddad K, Gervaix A, et al. A Mobile Device App to Reduce Medication Errors and Time to Drug Delivery During Pediatric Cardiopulmonary Resuscitation: Study Protocol of a Multicenter Randomized Controlled Crossover Trial. JMIR Res Protoc [Internet]. 2017 Aug 22 [cited 2019 Oct 13];6(8):e167. Available from: http://www.ncbi.nlm.nih.gov/pubmed/28830854
7. GitHub—sagieppel/Fully-convolutional-neural-network-FCN-for-semantic-segmentation-Tensorflow-implementation [Internet]. [cited 2020 Jan 18]. Available from: https://github.com/sagieppel/Fully-convolutional-neural-network-FCN-for-semantic-segmentation-Tensorflow-implementation
8. [1708.08711] Setting an attention region for convolutional neural networks using region selective features, for recognition of materials within glass vessels [Internet]. [cited 2020 Jan 19]. Available from: https://arxiv.org/abs/1708.08711

What is claimed is:

1. A medication delivery device comprising:
(a) an elongate member having a centrally disposed fluid and pressure channels;
(b) a plurality of fluid cartridge adapters spaced along the length of the elongate member, wherein each adapter is adapted to receive fluid from a pre-filled removable cartridge;
(c) a fluid channel that is centrally disposed within the elongate member and fluidly linked to the fluid cartridge adapters, wherein the fluid channel's first end is operably connected to fluid cartridge adapters and can be enclosed in the fluid cartridge while the second end is operably linked to an exit portal;
(d) a pressure channel that is centrally disposed within the elongate member and pressureably linked to the fluid cartridge adapters, wherein the fluid channel's first end is enclosed in the fluid cartridge while the second end is operably linked to a hole to connect to one or more pressure modifying devices and one or more pressure controllers;
(e) a hole that connects to one or more pressure modifying devices and one or more pressure controllers;
(f) one or more pegs that fit into a hole of the elongate member to restrict the elongate member's movement and pressureably link to the fluid cartridge adapters;
(g) one or more one-way valves to prevent fluid from entering the removable cartridge and moving away from the fluidly linked exit portal to the patient, syringe, or similar device;
(h) one or more structural housings that can slide freely vertically up and down or horizontally side to side to contact the elongate member and restrict the elongate member's movement;
(i) a plurality of cartridge holders attached to the structural housing and dimensioned so as to receive the removable cartridges and restrict the movement of the cartridges from a substantially upright position, wherein a turning motor to facilitate the presentation of the cartridge to a machine-readable indicator or sensor.

2. The medication delivery device of claim 1, wherein the device further comprises a computer that can receive input from an external computer, software, database, CPOE, EHR, or CDSS by a user who enters commands to control the amount of fluid that is released by changing the amount or length of time that pressure is applied to one or more removable fluid cartridges.

3. The medication delivery device of claim 2, wherein the computer receives output from optical machine-readable representation of data and fluid level and volume of pre-filled removable cartridges by either a camera or scanner.

4. The medication delivery device of claim 2, wherein the computer further verifies the credentials of the user who may be a pharmacist, pharmacist technician, nurse, physician, or other qualified personnel to handle cartridges according to Drug Classifications, Schedule I, II, III, IV, V.

5. The medication delivery device of claim 2, wherein the computer permits the creation of rules for custom BUD based on individual usage patterns, recalled lots as directed by the manufacturer, hospital, government, or other regulatory body, and usage logs that suggest that the cartridge has been previously used.

6. The medication delivery device of claim 2, wherein the computer references an external database or external databases to access additional information about the cartridge and its contents including, but not limited to, package description, product type name, proprietary name, concentration, volume, package size, and DEA schedule.

7. The medication delivery device of claim 2, wherein the computer comprises a computer that can receive input or output and is networked to and from an information processor, remote server over a network, database, database client, printer, and scanner.

8. The medication delivery device of claim 2, wherein the computer is networked to one or more information storage devices.

9. The medication delivery device of claim 1, wherein the device further comprises a plurality of pre-filled removable cartridges that have a plurality of machine-readable indicators each labeled on a face of the cartridge with a machine-readable indicator wherein the machine-readable indicator comprises an optical machine-readable representation of data consisting of name of fluid, concentration, volume, lot number, dosing parameters, instructions, warnings, usability, Drug Classifications, Schedule I, II, III, IV, V, manufacturer expiration date, BUD, manufacturer date, storage and handling conditions, location, reordering, just-in-time inventory strategy, supply chain management, expenses, billing, among others.

10. The medication delivery device of claim 1, wherein the device further comprises one or more pressure modifying devices that are pressureably linked to the fluid channel so that a change in pressure from the pressure modifying device causes fluid to move into the fluid channel.

11. The medication delivery device of claim 1, wherein the device further comprises one or more pressure controllers consisting of a pressure sensor and variable orifice valve to control pressure with in pre-filled removable cartridges and air and fluid channels.

12. The medication delivery device of claim 1, wherein the device further comprises remote server references a fill list to refer the type and quantity of each medication in the pre-filled removable cartridges including expected contents, excess, and shrinkage.

13. The medication delivery device of claim 12, wherein the remote server references rules or user input data to accept or reject cartridges.

14. The medication delivery device of claim 12, wherein the remote server verifies that the machine-readable indicator was created by said medication delivery device or digital signature to ensure the provenance on the machine-readable indicator.

15. The medication delivery device of claim 1, wherein the device further comprises an information processor to record the time, date, volume dispensed, volume remaining, volume waste, usage of cartridge, and remainder, or waste, location of cartridges, among other information about the cartridges to track current inventory on hand of medications throughout a health care facility to support supply chain management and just-in-time (JIT) inventory strategy.

16. The medication delivery device of claim 1, wherein the device further comprises a database client to generate reports for inventory and supply chain management, reordering, usage, billing, among other reasons.

17. The medication delivery device of claim 1, wherein said database client can accept pricing data from a database Medicaid or others to provide data to the user or calculate inventory information including inventory turnover, inventory turnover days, basic stock, safety stock, order cycle time, just-in-time inventory, among others.

18. A method for controlling use of the device of claim 1, comprising:
(1) prompting a user to enter information about personnel and patient information;
(2) prompting a user to enter a medication order into an external computer to program medication delivery device;
(3) outputting user entered and other relevant patient information;
(4) detecting a machine-readable indicator on the pre-filled removable cartridge by a scanner when the cartridge is inserted into the device, wherein the machine-readable indicator comprises an optical machine-readable representation of data;
(5) accessing an information processor to interpret the machine-readable indicator and perform various rules to identify cartridges that have been recalled, expired, or previously used and perform security checks such as verifying that the cartridge barcode was originally generated;
(6) detecting the fluid level and volume of the pre-filled removable cartridge by a scanner when the cartridge is inserted into the device, wherein the machine-readable indicator comprises an optical machine-readable representation of data
(7) connecting to a remote server over a network or operatively connected to the Internet to compare a fill list to the expected data and data scanned from a machine-readable indicator or plurality of machine-readable indicators to determine discrepancies, sign digitally to verify the validity of the barcode for recall, manufacturer expiration date, user-generated BUD, prior usage and access fill lists from which inventory of cartridges can be sorted and built upon;
(8) using an algorithm that is programmed into the computer to calculate a proper dose for administering to the patient based on the information outputted to the computer by scanner and the user inputted information about the patient; and
(7) administering the calculated dose by one or more pressure modifying devices that can exert positive pressure on the fluid contained in the cartridges and one or more pressure controllers, including a pressure sensor and a variable orifice valve in each, that can control positive pressure on the fluid contained in the cartridges, that can be controlled by an external computer or software from orders placed by health care personnel or database such as a CPOE and EHR then present information related to the scanner's output on a display.
